(19) United States
(12) Patent Application Publication
(10) Pub. No.: US 2010/0170483 A1
(43) Pub. Date: Jul. 8, 2010

(54) FILM RESISTOR IN AN EXHAUST-GAS PIPE

(75) Inventors: Karlheinz Wienand, Aschaffenburg (DE); Matthias Muziol, Mainhausen (DE); Karlheinz Ullrich, Grossumstadt (DE)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: HERAEUS SENSOR TECHNOLOGY GMBH, Hanau (DE)

(21) Appl. No.: 12/597,628

(22) PCT Filed: Apr. 23, 2008
(86) PCT No.: PCT/EP08/03236

§ 371 Date: Oct. 26, 2009

(30) Foreign Application Priority Data
Apr. 26, 2007 (DE) 10 2007 020 084.8

(51) Int. Cl. F02M 25/07; G01P 13/00; G01F 1/00; G01N 33/00; B08B 7/00; B32B 37/00; G01K 13/00; G01R 27/08; H01C 1/024; B23P 11/00

(52) U.S. Cl. 123/568.12; 73/170.01; 73/861; 73/23.31; 134/42; 156/293; 374/148; 324/691; 338/230; 29/611

(57) ABSTRACT

The invention relates to the arrangement of a film resistor (chip) in an exhaust-gas pipe or exhaust-gas recirculation pipe, wherein the film resistor is fixed in a carrier, which is sealed against a shield or a housing, wherein this shield or housing is connected tightly to the exhaust-gas pipe or exhaust-gas recirculation pipe, wherein the carrier and the shield or the housing are sealed against each other spaced apart from the exhaust-gas pipe or exhaust-gas recirculation pipe radially outside of this pipe.

The present invention relates to the arrangement of film resistors (chips), particularly for anemometers or soot sensors or temperature sensors, in pipes with hot fluids, particularly exhaust-gas pipes, as well as measurement devices for measurements in hot fluids with film resistors and an exhaust-gas recirculation with a hot-film anemometer. Film resistors are commercially available according to the Heraeus brochure PTM-W2.

DE 102 60 896 discloses a film resistor (chip) for anemometric measurements, wherein this film resistor projects into an exhaust-gas pipe.

DE 199 59 854 discloses film resistors (chips) that project into an exhaust-gas pipe and that are fixed in a carrier connected to the exhaust-gas pipe. DE 199 59 854 describes an exhaust-gas recirculation in which the incoming air is measured with a flow mass sensor according to the anemometric principle, and a second flow mass sensor is arranged in the exhaust-gas channel after a water cooler for the measurement of the exhaust-gas quantity.

The anemometric measurement principle of a flow quantity sensor is known from DE 195 06 231.

DE 103 05 694 discloses a flow mass sensor for exhaust-gas measurements in which the chip parts are produced from thin metal films. These sensors are susceptible to noise and possibly deliver non-reproducible results.

In DE 10 2006 058 425, in the case of a hot-film anemometer, a heat and temperature measurement element is arranged in a ceramic disk or a metal disk.

The object of the present invention comprises providing a temperature-change-resistant connection between the ceramic chip and the exhaust-gas pipe, wherein this connection is suitable in terms of complexity, material, and costs for mass production. The susceptibility of sensor-controlled exhaust-gas recirculation systems is to be minimized and their sensitivity is to be maximized.

To achieve the object, the carrier of the film resistor (chip) is sealed against a shield or a housing outside of the exhaust-gas pipe or exhaust-gas recirculation pipe.

This allows an arrangement in which carriers are fixed in the metallic housing outside of the exhaust-gas pipe, particularly between the exhaust-gas recirculation pipe and the seal of the carrier against the housing.

Furthermore, according to the invention it is possible to space the shield from the carrier within the exhaust-gas pipe, particularly the exhaust-gas recirculation pipe.

The solutions of the object are described in the independent claims. Preferred embodiments are described in the dependent claims.

In preferred embodiments, the current feedthrough for the film resistor is sealed relative to the medium in the exhaust-gas pipe and insulated electrically against the exhaust-gas recirculation pipe and the exhaust-gas pipe. For this purpose it is particularly advantageous to seal the film resistor tightly in the region of its fixing drop to the ceramic carrier, because in this way the connection wires are separated from the exhaust gas and falsification of a measurement by exhaust gas in the region of the connection wires is excluded. The problem of the falsification of measurements lies in the fact that, among other things, there is loss of insulation or the appearance of parallel resistance paths, for example due to deposits or moisture.

In one arrangement of a film resistor (chip), in particular a film resistor of an anemometer in a pipe with hot fluid, in particular an exhaust-gas pipe or exhaust-gas recirculation pipe, wherein the film resistor (chip) is fixed in a carrier, in particular a hollow body, which is sealed against a shield or a housing that is connected tightly to the exhaust-gas pipe or exhaust-gas recirculation pipe, according to the invention, the carrier is sealed against the shield or the housing outside of the exhaust-gas pipe or exhaust-gas recirculation pipe.

In particular, a measurement device comprises the film resistor, the carrier, the shield, as well as the seal between the carrier and the shield.

This allows measurements with a film resistor (chip) within a hot fluid over 500° C. in a metal pipe over its service life, when the film resistor (chip) is fixed in a carrier that is sealed against a metal housing outside of the metal pipe and, according to the invention, the metal housing is connected tightly to the exhaust-gas pipe or exhaust-gas recirculation pipe.

A measurement device suitable for measurements in hot fluids with a film resistor, a carrier, in particular a hollow body, in which the film resistor is fixed mechanically, and a shield or a metallic housing, wherein the shield or the metallic housing is sealed against the carrier, features, according to the invention, arranged in the longitudinal direction of the carrier, the electrical feed lines for the film resistor, wherein the film resistor is mainly spaced apart along the carrier length from the seal that holds the carrier tightly against the shield or the housing. According to the invention, the spacers used for spacing the carrier relative to the housing no longer need to be air-tight. The high complexity for the tightness due to the temperature load can be spared according to the invention. The feed lines comprise the connection wires of the film resistor and possibly additional extensions. The invention also extends to film resistors with connection surfaces instead of connection wires. In this case, the feed lines are not components of the film resistor.

In preferred embodiments:
a film resistor is fixed in an opening of a hollow body,
the hollow body is fixed in the metallic housing,
the carrier is sealed against the metal housing on its end facing away from the film resistor,
the metal housing is spaced apart from the carrier, in particular a ceramic, multi-hole capillary tube, in particular the shield is spaced apart from the carrier within the exhaust-gas pipe or exhaust-gas recirculation pipe,
the metal housing encloses the carrier, in particular a ceramic multi-hole capillary tube, on the side of the film resistor up to feedthroughs for the film resistor or the film resistors,
the carrier, in particular a ceramic multi-hole capillary pipe, is fixed to the metal housing in a region between the film resistor and the seal,
the measurement device has two carriers, in particular ceramic multi-hole capillary pipes in each of which a film resistor is fixed,
the film resistor is sealed gas-tight against the carrier in the region of the feedthrough,
the gas-tight seal is made of inorganic material,
the carrier and film resistor are made at the position of the seal from materials adapted to each other with respect to expansion.

In a simple embodiment the carrier is constructed as a pipe.

A preferred measurement device is an anemometric measurement device of a flow meter containing film resistors, wherein, according to the invention, two film resistors are each fixed in a pipe that is adapted in terms of expansion, so that the expansion coefficients of the ceramic chip and the pipe carrying this chip deviate by a maximum of ±2.5×10⁻⁶/K. Materials adapted in terms of expansion allow a seal with inorganic material, for example glass. For materials that have not been adapted in terms of expansion, glass has elasticity properties that are too low, and the known elastic seal materials exhibit heat resistance that is too low.

In the case of one method for anemometric measurement, in particular in exhaust-gas recirculation pipes, according to the invention, the anemometric measurement is performed with a film resistor in the exhaust-gas recirculation pipe, wherein the carrier of the film resistor is led through the exhaust-gas pipe and is sealed against a metallic housing on its side located outside of the exhaust-gas pipe.

In one arrangement of a measurement device in an exhaust-gas recirculation pipe for anemometric measurement, in particular in exhaust-gas recirculation pipes, according to the invention, the anemometric measurement is performed with a film resistor in the exhaust-gas recirculation pipe, wherein the carrier of the film resistor is led through the exhaust-gas pipe and is sealed against a metallic housing on its side located outside of the exhaust-gas pipe.

In the case of one method for the production of an anemometric measurement device, comprising a heating device that possibly has one or two additional measurement resistors and possibly an additional measurement resistor that possibly has an additional heating resistor, the film resistor constructed as a heater is fixed in a hollow body whose expansion coefficient differs from that of the film resistor by no more than 2.5×10⁻⁶/K, in particular by a maximum of 1×10⁻⁶/K, wherein, according to the invention, the hollow body is fixed in a metallic housing, so that it remains spaced apart from the metallic housing up to the attachment. Preferably, the hollow body is also sealed against the metal housing on the end of the pipe opposite the film resistor.

For the production of a measurement device, in particular anemometric measurement device, according to the invention a hollow body, on which a film resistor is fixed, is sealed with an organic seal material against the metal housing on its end opposite the film resistor.

For the production of a measurement device that projects into an exhaust-gas pipe or an exhaust-gas recirculation pipe and that has a film resistor fixed in a hollow body that is, in turn, fixed in and spaced apart from a metal housing this attachment of the hollow body to the metal housing is arranged, according to the invention, outside the exhaust-gas pipe.

For use of a metallic housing for the heat insulation of a measurement device against hot exhaust gases, the metallic housing is spaced apart from a hollow body that carries a film resistor on its end projecting into the exhaust-gas pipe. The hollow body is advantageously fixed to the metallic housing outside of the pipe with the hot exhaust gases.

The measurement device according to the invention is suitable for use as an anemometric measurement device or as a soot sensor or as a temperature sensor.

For each arrangement or measurement device, several film resistors could be arranged in a carrier or several carriers could be used for several film resistors, for example in an anemometer measurement device with a film resistor as a heating element and another film resistor as a temperature sensor. The chip with the heating element preferably has, in addition, one, in particular two measurement resistors with which the direction of the gas flow can be determined. Furthermore, the temperature sensor preferably has a heater for clean burning.

The measurement of the flow masses is performed according to an embodiment according to the invention neither in a region cooled with water nor after a region cooled with water. For this embodiment, the hot-film anemometer is arranged before the cooler or within a cooler cooled with air.

According to the invention, the flow quantity of suctioned air no longer needs to be determined.

With the determined parameters of the vehicle internal combustion engine, an optimization of the engine operation is achieved with respect to low environmental burden and efficiency, wherein the parameters are determined in a very simple way.

Exhaust gas discharged from the engine is fed to a hot-film anemometer, wherein an actual value signal (X, X1, X2) is formed from the determined flow quantities of recirculated exhaust gas and possibly suctioned air and is compared with a desired value signal (w) for an optimal operating point of the engine, wherein a control deviation of the actual value signal leads to a control signal that acts on the exhaust-gas recirculation with the help of a control element.

With the help of a control element (Y, Y1), at least one control element in the exhaust-gas recirculation is driven, which element is preferably constructed as a controllable valve.

In a preferred embodiment of the method, the desired-value signal (W) is derived from a power default signal (e.g., gas pedal) and at least one parameter of the engine. As a parameter, a signal has proven effective that is formed from the exhaust-gas temperature or the rotational number as well as the mass of the recirculated exhaust gas. Here, at least one signal is compared as an engine parameter with a reference signal by the formation of a difference value, wherein the exceeding of a given difference value leads to a control signal (Y, Y1, Y2).

While the control signals Y, Y1 act directly on the exhaust-gas recirculation—preferably by a controllable valve—the control signal Y2 acts on a control element that is located in the inlet region of the internal combustion engine, into which flows a mixture of incoming air and partially recirculated exhaust gas.

In one embodiment of the method, in the hot-film anemometer, the flow passes through at least two heating resistors held at a constant temperature one after the other for measuring the flow mass of the exhaust gas; here, an adjustable flow (I₁, I₂) passes through each of the heating resistors, wherein a signal for the flow mass and its direction is formed from the intensity of the currents (I_{I}, I₂).

Furthermore, in one embodiment of the method, the resultant flux of the mass flow is determined from at least one pulsating flow I_{I}, I₂ by alternating multiplication of the flow amplitude with +1 and −1 and a subsequent formation of the difference value.

In the case of a device for (partial) exhaust-gas recirculation from an outlet region of a vehicle internal combustion engine into an air inlet region, into which can be fed a mixture of exhaust gas and incoming air of the engine, wherein this mixture can be adjusted by a regulator, for determining the mass of the recirculated exhaust-gas air, according to the invention a hot-film anemometer is arranged in the exhaust-gas recirculation channel before the cooling system or in an air-cooled cooler.

The outlet region is connected to an inlet region of the internal combustion engine via a flow channel for the exhaust-gas recirculation, which has a controllable valve as a control element and the hot-film anemometer.

In this way, a fuel quantity can be adjusted as a function of the air mass flow rate under consideration of a power default signal.

In a first preferred embodiment of the device, the hot-film anemometer has at least one measurement resistor and at least one heating resistor, wherein the resistors are each formed with micro-system technology. Advantageously, the resistors are formed as thin-film or thick-film elements based on the measurement technology of platinum or a platinum-group metal.

The heating resistor or the heating resistors are provided for operation in the temperature range of 500 to 750° C.

A mass flow sensor with a measurement resistor of short response time and a quick-acting micro-heater is known, for example, from EP 0 964 230 A2.

In a second advantageous embodiment of the invention, the hot film anemometer has at least two quick-acting micro-heaters or heating resistors that are operated at well-defined, fixed excess temperatures, e.g., 450° C. and 550° C. The temperatures are selected so that accumulating soot is always combusted by pyrolysis and the micro-heaters thus always remain clean. Quick-acting control electronics supply the heaters by current, so that their temperatures are held constant. The evaluation of the heating currents can permit a unique conclusion to be reached both on the mass flow and also on the mass temperature. Through the use of platinum heating elements that can be produced both in thick-film and also in thin-film technology, platinum-heating temperatures can be set from 500° C. to 750° C. through the use of the well-defined resistor-temperature characteristic curve.

One essential advantage of the second embodiment (two heating resistors) is to be seen in that practically no accumulation of soot takes place on the heating resistors, so that they are always operated with optimal measurement characteristics.

The heating or measurement resistors are advantageously constructed in at least two track conductors on a plate-shaped membrane—preferably made of an electrically insulating and heat-resistant material as, for example, ceramic.

In an inventive refinement, hot-film anemometers are arranged suitably for mass production and hot-film anemometers that are suitable for mass production are provided for exhaust-gas recirculation, which, in particular self-cleaning, hot-film anemometers counteract drift or functionally stable flow sensor elements exposed to strong contamination as, e.g., exhaust gas.

One significant aspect for the present invention is a self-cleaning of the temperature measurement element through annealing by a heating conductor. In particular, this heating conductor is integrated on the chip side of the temperature measurement element. In a preferred embodiment, at least two platinum thin-film resistors are arranged on a ceramic carrier plate. This allows a heating of the temperature measurement element for baking or annealing contaminants.

In particular, the two resistors of the temperature measurement element are arranged on a ceramic substrate, preferably on a solid ceramic plate.

As ceramic components of a multi-part, ceramic element, in addition to the carrier part that is advantageously already composed as a laminate, the temperature measurement element and the heating element are also to be taken into account. In a very particularly preferred way, the carrier part is constructed as a cover or as one side or face of a hollow body, in particular its end face. Instead of a ceramic carrier, the resistors could also be arranged on the ceramic substrate on an alternative carrier.

It is advantageous when the temperature measurement element has rectangular, ceramic carrier plates with two long and two narrow edges and that the ceramic carrier plates are arranged in the region of one of the narrow edges between the ceramic films of the ceramic film laminate or between at least two parts of the ceramic component.

Likewise, it is advantageous when the one or more heating elements have rectangular, ceramic carrier plates with two long and two narrow edges and that the ceramic carrier plates are arranged in the region of one of the narrow edges between the ceramic films of the ceramic film laminate or between at least two parts of the ceramic component.

In a very particularly preferred way, the temperature measurement element or the at least one heating element has a rectangular ceramic carrier plate with two long and two narrow edges, wherein the ceramic carrier plates are arranged in the openings of a cover or a hollow body end face.

The platinum thin-film resistors are here arranged advantageously on the end of the carrier plate facing away from the ceramic film laminate or the ceramic components, in order to guarantee the lowest possible thermal influence of the platinum thin-film resistors through the thermally inert ceramic film laminate or the thermally inert ceramic components.

In order to prevent a mutual influence of the temperature measurement element and heating element, it is advantageous when the platinum thin-film resistor of the heating element is arranged farther removed from the ceramic film laminate or from the ceramic component than the platinum thin-film resistor of the temperature measurement element. In this way, the platinum thin-film resistors of the heating element are not arranged in the same flow stream of the measurement medium as the platinum thin-film resistors of the temperature measurement element.

According to the invention, in a particularly preferred way, an anemometric measurement device is also provided in which film resistors are fixed in a cover or a hollow body in an opening or openings of the cover or hollow body, wherein two resistors differ by one or three orders of magnitude.

The resistor that is larger by one to three orders of magnitude is suitable as a temperature measurement resistor and is designated below as such. The resistors that are smaller by one to three orders of magnitude relative to the temperature measurement resistor are used for heating. With respect to these heating resistors, in the scope of the present invention, various functions are differentiated:

1. Heating resistors for self cleaning of the temperature sensor as a component of the temperature sensor.
2. Heating resistors as thermal output sensors for determining a mass flow according to the anemometric principle.

Thermal output sensors with two heat conductors allow the determination of the direction of the mass flow. Thermal output sensors with an additional temperature measurement resistor allow an exact temperature setting of the thermal output sensor. The present invention here relates exclusively to film resistors that are constructed as a thick film or thin film, preferably in platinum, in particular as a platinum thin film. The film resistors are arranged on a substrate, in particular on a ceramic substrate. The ceramic substrate could be constructed as a carrier or arranged on a carrier as, e.g., a metal plate. In common usage, film resistors deposited on a carrier material are likewise designated as film resistors, so that, in terms of language, film resistors in the narrow sense as a pure resistive film are not differentiated from film resistors including the carrier material. The film resistors placed in openings of a cover or hollow body include the substrate on which the thin film or thick film is arranged as a resistive film.

In one preferred embodiment, the film resistors are arranged in the narrow sense on a ceramic substrate. Different film resistors in the broad sense can be arranged one next to the other in an opening of a cover or hollow body or else separately each in one opening. Preferably, thermal output sensors and temperature sensors are spaced apart from each other. Two heat conductors of one thermal output sensor are preferably arranged one behind the other, so that they lie one behind the other in the direction of flow. Preferably, thermal output sensors are constructed with two heat conductors on a common substrate or with two identical chips arranged one after the other.

The openings of the cover or hollow body are expediently slots or boreholes.

The cover is provided for the tight closure of a pipe. If the cover is made of metal, then it can be fused with a metal pipe. The film resistors in the broad sense are guided through the opening or the openings of the cover and are fixed in the opening or in the openings on the cover. The hollow body is used for holding the connections of the film resistors, whose sensitive part projects through the opening or the openings out of the hollow body.

One significant aspect of the present invention is that resistors generated using thick films or thin films are integrated to form a sensor element that can be easily installed in an exhaust-gas channel during mass production. The solution according to the invention to place film resistors in a cover or hollow body allows a simple seal of the cover or hollow body both relative to the carrier material of the resistors and also to the material of the exhaust-gas channel.

According to the invention, it is achieved that the film resistors can be constructed perpendicular to the base surface of a cover or hollow body. From this are produced production-related advantages relative to an arrangement continued parallel to a plate. Here, the invention is not restricted to a perpendicular embodiment, but instead allows any angle to the surface of the cover or hollow body. As an essential inventive advantage, the vertical components can be constructed at angles according to the present invention. Accordingly, the advantage of the present invention occurs particularly for angles of 60 to 90 degrees, particularly of 80 to 90 degrees.

In preferred embodiments
the hollow body is constructed as a pipe open on one side, particularly as a pipe closed on one side;
the cover is constructed as a disk;
the base surface of an opening for holding at least two film resistors is smaller by at least one order of magnitude than the cover base surface or a corresponding hollow body base surface;
the cover or the hollow body has two openings for holding film resistors;
the cover is made of ceramic material;
the film resistors held on ceramic carrier material are fixed in the opening of a ceramic cover, in particular a ceramic disk with glass solder;
the film resistors carried on a ceramic substrate are fixed in at least one opening of a metal cover or hollow body, particularly a metal disk fused on a metal pipe with a sealing compound or glass;
the two resistors of the temperature measurement element lie in one plane,
the smaller resistor (heater 202d) frames the larger resistor (202a for temperature measurement).

The measurement device according to the invention is suitable for flow sensors or soot sensors.

The hot-film anemometer is operated with the thermal output resistor and the temperature sensor according to the anemometric principle. According to the invention, the temperature sensor is equipped as part of an anemometric measurement device with another heat conductor. This construction allows the cleaning of the temperature sensor by annealing with the heater. In the anemometric measurement device, it has proven effective to decouple, advantageously to space apart, the temperature sensor and the thermal output sensor to be distinguished from the heater of the temperature sensor, in particular to place them in separate openings of the cover or hollow body. The temperature sensor has a significantly higher resistance than the heater, typically one to three orders of magnitude higher. With the temperature sensor, the influence of the temperature of the exhaust gas on the determination of the flow mass can be corrected.

A temperature measurement resistor that is possibly arranged on the thermal output sensor and with which the temperature of the heat conductor can be adjusted in an particularly precise way is to be distinguished from the temperature sensor. In contrast with the temperature sensor, a complete temperature measurement resistor is not provided for the measurement of the fluid temperature, because it is suitable during the operation of the thermal output sensor only for its temperature control.

The thermal output sensor and the temperature sensor preferably both have a heat conductor and a temperature measurement resistor. If the thermal output sensor and temperature sensor are structurally identical, then their functional determination is determined by the electronics.

It has proven effective to form the carrier of the platinum thin-film resistors as a thin plate, so that an extremely low thermal inactivity of the system and thus a high response rate of the platinum thin-film resistors are produced. For forming a ceramic composite, sintered ceramic films can be used, that are then preferably bonded with a glass solder. The materials used for the construction of the hot-film anemometer can be used with excellent results at temperatures in the range of −40° C. to +800° C.

It is particularly preferred when the ceramic carrier plates have a thickness in the range of 100 μm to 650 μm, particularly 150 μm to 400 μm. Al₂O₃ has proven effective as the material for the ceramic carrier plate, particularly with at least 96 wt. % and advantageously greater than 99 wt. %.

For the platinum thin-film resistors, it has proven effective when these each have a thickness in the range of 0.5 μm to 2 μm, in particular 0.8 μm to 1.4 μm. Heating resistors preferably exhibit 1 to 50 Ohm and tend toward lower values with reduction in size of the components. In the case of currently common dimensions of components, 5 to 20 Ohm are preferred. Temperature measurement resistors preferably exhibit 50 to 10,000 Ohm and likewise tend toward lower values with reduction in size of the components. In the case of current common dimensions of the components, 100 to 2000 Ohm are preferred. On the temperature chip, the temperature measurement resistor is greater by a multiple than the heating resistor. In particular, these resistors differ by one to two orders of magnitude.

In order to protect the platinum thin-film resistors from corrosive attack by the measurement medium, it has proven effective when these are each covered with a passivation film. The passivation film here advantageously has a thickness in the range of 10 μm to 30 μm, in particular 15 μm to 20 μm. A passivation film made of at least two different individual films, in particular individual films made of Al₂O₃ and glass ceramic, has proven particularly effective. The thin-film technology is suitable for creating the preferred film thickness of the Al₂O₃ film of 0.5 μm to 5 μm, in particular 1 μm to 3 μm.

The platinum thin-film resistors are here preferably arranged on the end of the carrier plate facing away from the cover or hollow body, in order to guarantee the smallest possible thermal influence of the platinum thin-film resistors by the thermally inert cover or hollow body.

In order to prevent a mutual influence of the temperature measurement element and heating element, it is advantageous when the platinum thin-film resistor of the heating element is arranged farther removed from the cover or hollow body than the platinum thin-film resistor of the temperature measurement element. Therefore, the platinum thin-film resistors of the heating element are not arranged in the same flow stream of the measurement medium as the platinum thin-film resistors of the temperature measurement element.

The preferred arrangement of the temperature measurement element is in front of the heating element in the direction of flow.

Preferably, the carrier plates of the heating element and the temperature measurement element are spaced apart from each other and, indeed, particularly parallel to each other.

It has proven effective particularly for the measurement of media with alternating direction of flow, when two heating elements and one temperature measurement element or two temperature measurement elements and one heating element are arranged in one row.

It has proven effective to arrange the carrier plates of the heating element and the temperature measurement element in the cover or hollow body spaced apart from each other and parallel to each other.

With the hot-film anemometer according to the invention, a mass flow measurement of gaseous or fluid media in pipe lines is possible, particularly when the carrier plates are arranged in the direction of flow of the medium.

Preferably, the carrier plates of the heating element and the temperature measurement element are spaced apart from each other and, indeed, particularly in series between two equal ceramic films or parts of the ceramic component.

Here it has proven effective when the ceramic film laminate is formed from two ceramic films or when the ceramic component is formed from two ceramic pipes, whose walls each have, in cross section, a half-moon profile.

It has proven effective, particularly for the measurement of media with alternating direction of flow, when one temperature measurement element, two heating elements, and one temperature measurement element are arranged in series.

Furthermore, arrangements have proven effective in which the ceramic film laminate is formed from three ceramic films.

Here it has proven effective particularly when the carrier plates of the heating element and the temperature measurement element are arranged spaced apart from each other by ceramic films and parallel to each other.

It is preferred to arrange a heating element between a first and a second ceramic film and a temperature measurement element between the second and a third ceramic film of the three ceramic films, wherein the heating element and the temperature measurement element are arranged one next to the other at the same height of the ceramic film laminate.

In addition, it has proven effective when a heating element is arranged between a first and a second ceramic film of the three ceramic films and that two temperature measurement elements are arranged between the second and a third ceramic film of the three ceramic films, wherein the heating element is arranged between the temperature measurement elements.

Furthermore, arrangements have proven effective in which the ceramic film laminate is formed from four ceramic films.

Here, it is preferred when a first temperature measurement element is arranged between a first and a second ceramic film of the four ceramic films and a second temperature measurement element is arranged between a third and a fourth ceramic film of the four ceramic films and that a heating element is arranged between the second and the third ceramic film, wherein the heating element and the temperature measurement elements are arranged one next to the other at the same height of the ceramic film laminate.

Furthermore, it is preferred when a first temperature measurement element is arranged between a first and a second ceramic film of the four ceramic films and a second temperature measurement element is arranged between a third and a fourth ceramic film of the four ceramic films and that a heating element is arranged between the second and the third ceramic film, wherein the temperature measurement elements are arranged one next to the other at the same height of the ceramic film laminate and the heating element is arranged offset to the temperature measurement elements.

The use of a hot-film anemometer according to the invention is ideal for the mass flow measurement of gaseous or fluid media through pipe lines, wherein the carrier plates are arranged parallel to the direction of flow of the medium.

Here, the hot-film anemometer according to the invention is suitable, in particular for the measurement of gaseous media with a temperature in the range of −40° C. to +800° C., such as the temperature of, for example, the exhaust gas of an internal combustion engine.

The self cleaning by the heating of the temperature measurement element is suitable particularly for sensors arranged in the exhaust-gas region of internal combustion engines, in particular diesel engines. Soot-contaminated sensors are quickly made completely functional again through heating, in particular annealing. Here, this self cleaning can be repeated as often as desired during the service life of an engine.

The arrangement of several temperature measurement elements and heating elements on the carrier element also ideally allows the identification of the direction of flow or changes in the direction of flow of a medium. In this respect, it is advantageous to use the hot-film anemometer according to the invention for the measurement of media with a direction of flow changing at time intervals.

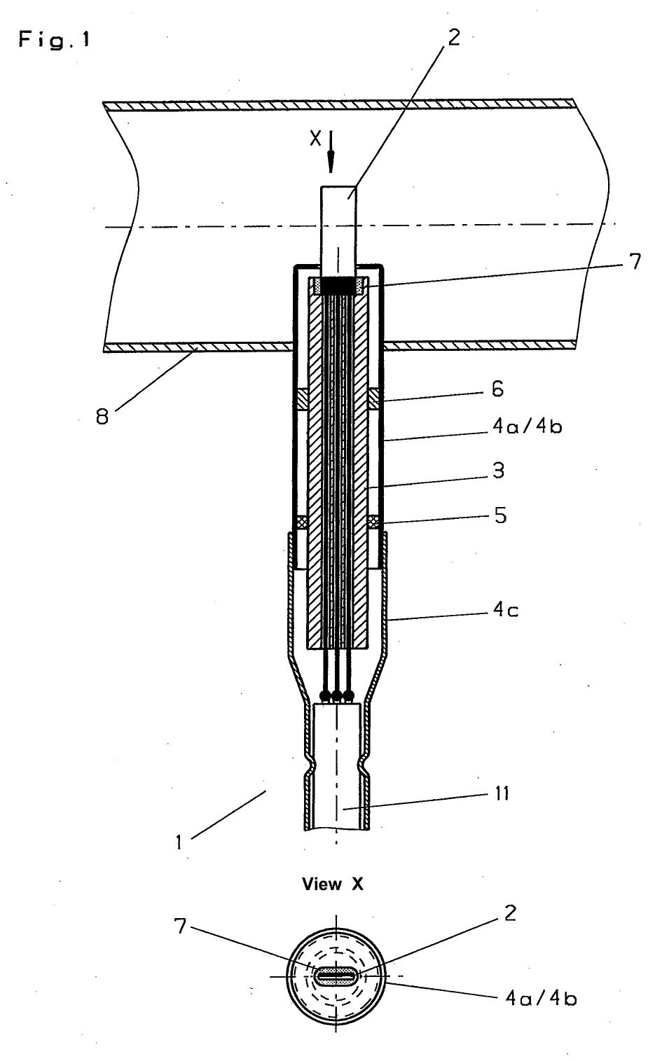

The subject matter of the invention is explained in greater detail below with reference to FIGS. 1 to [24b].

FIG. 1 shows a longitudinal section through an anemometric measurement device with associated top view,

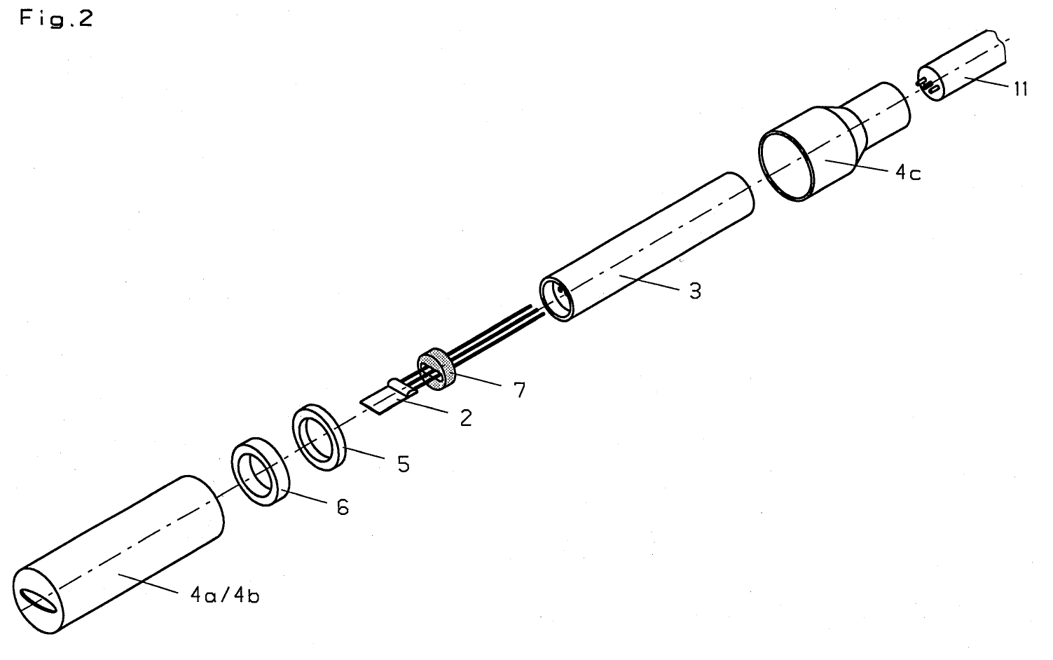

FIG. 2 shows the arrangement of FIG. 1 in a perspective, exploded view,

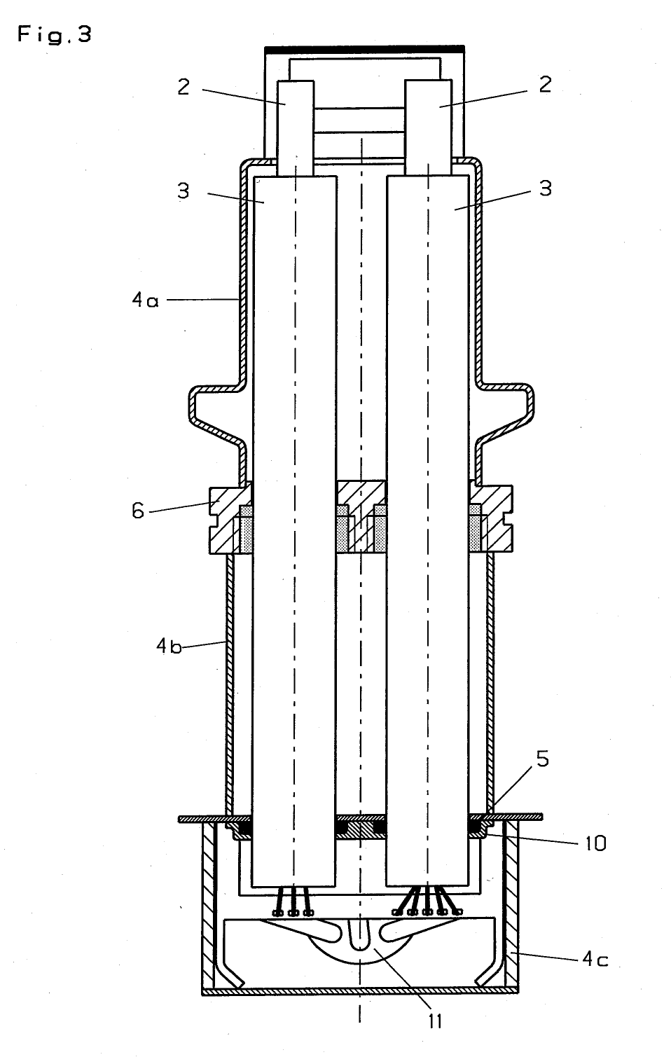

FIG. 3 shows a longitudinal section through an anemometric measurement device,

FIG. 3 shows a preferred anemometric measurement device with a carrier for each film resistor,

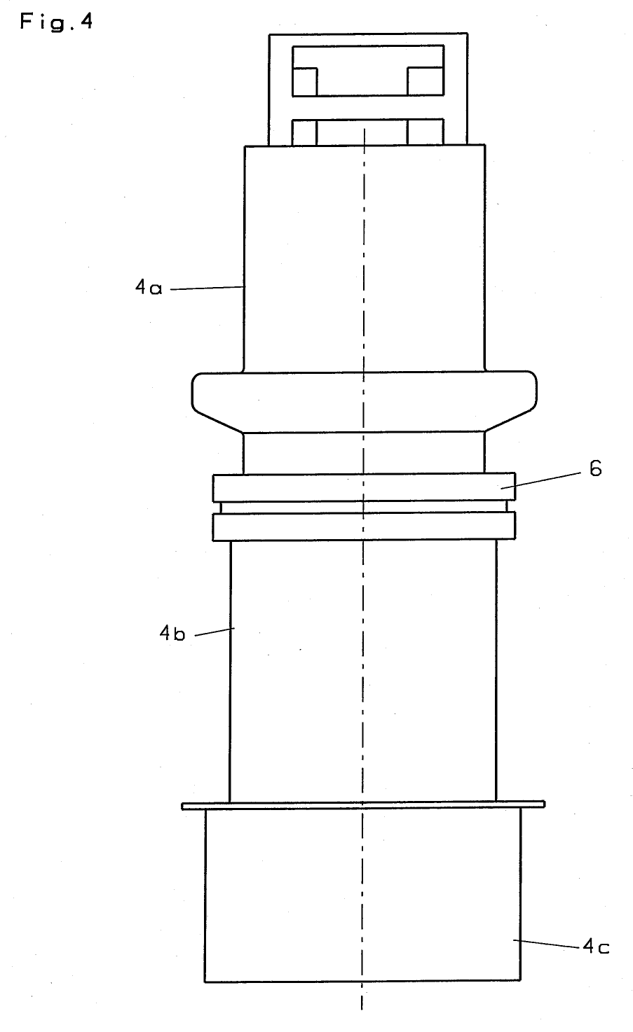

FIG. 4 is the outside view of FIG. 3,

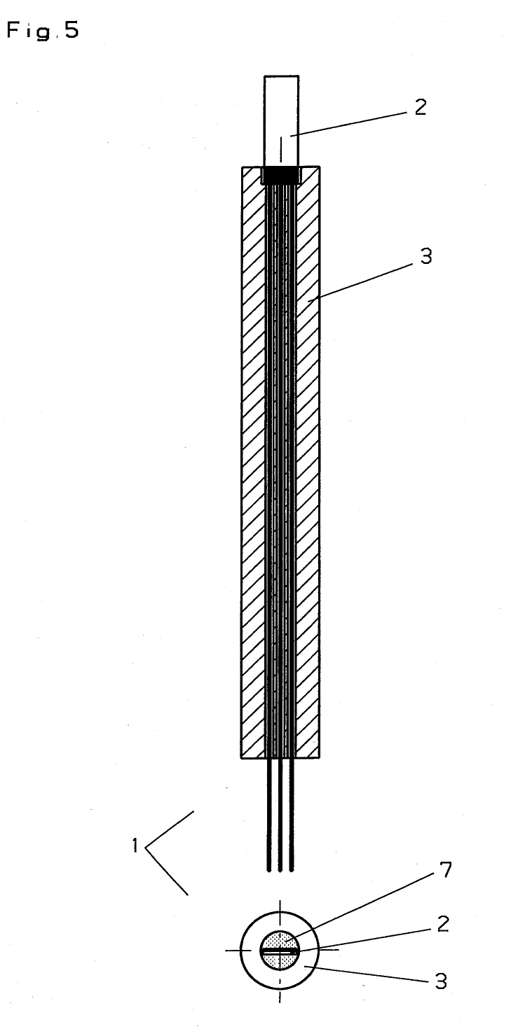

FIG. 5 shows a carrier from FIG. 3,

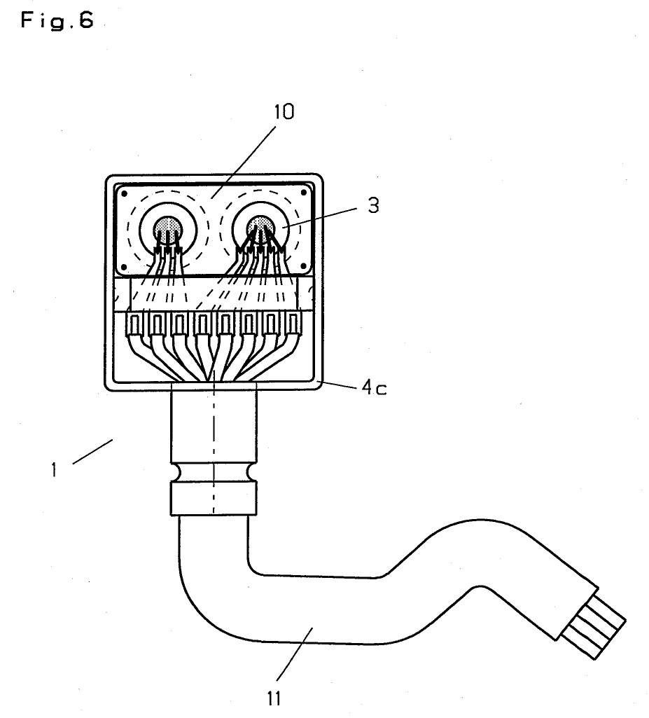

FIG. 6 shows the connection region of FIG. 3,

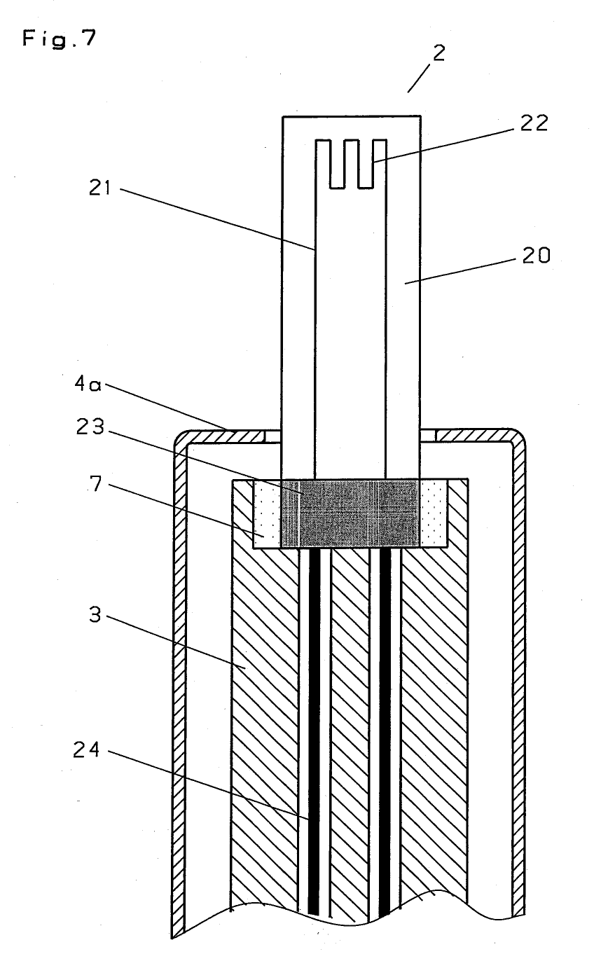

FIG. 7 shows a film resistor and its seal in the hot region of the measurement device,

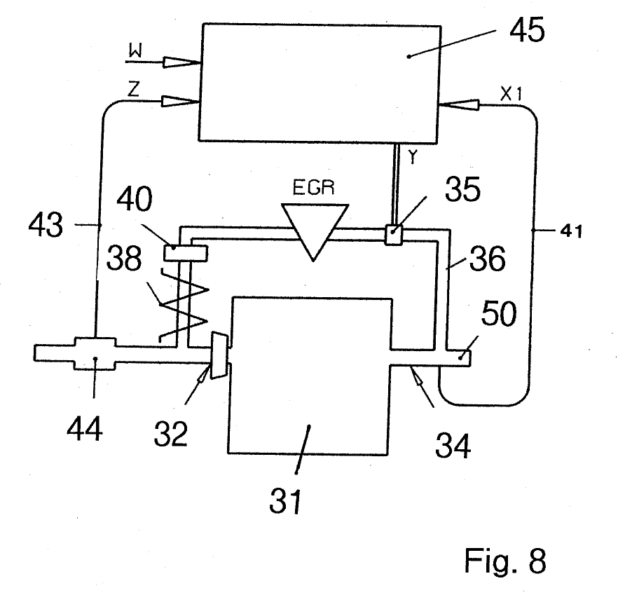

FIG. 8 shows schematically the configuration of a control loop for an internal combustion engine with exhaust-gas recirculation,

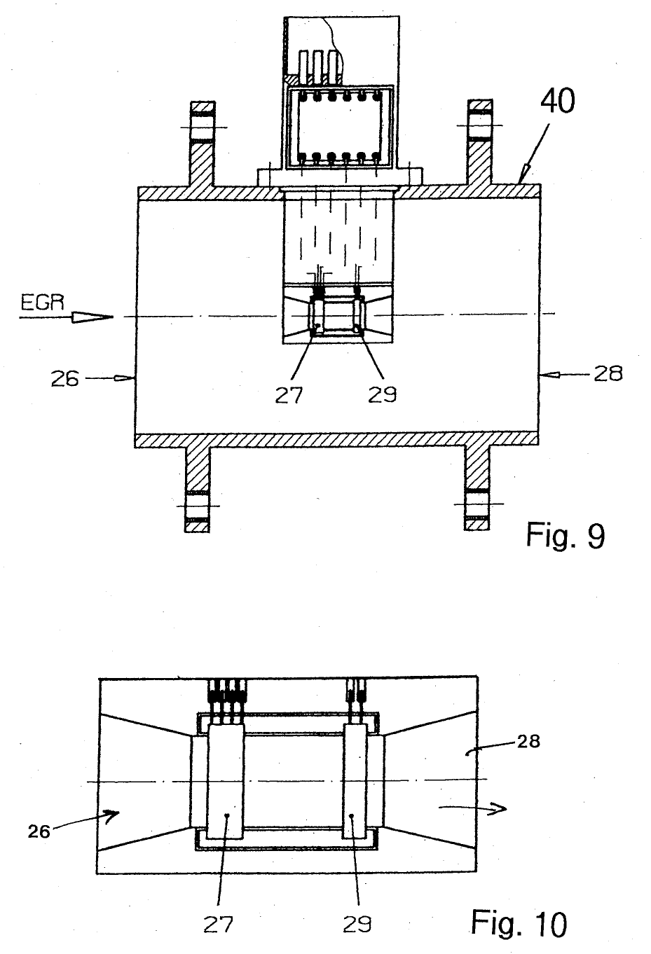

FIG. 9 shows the hot-film anemometer for the exhaust-gas recirculation in longitudinal section,

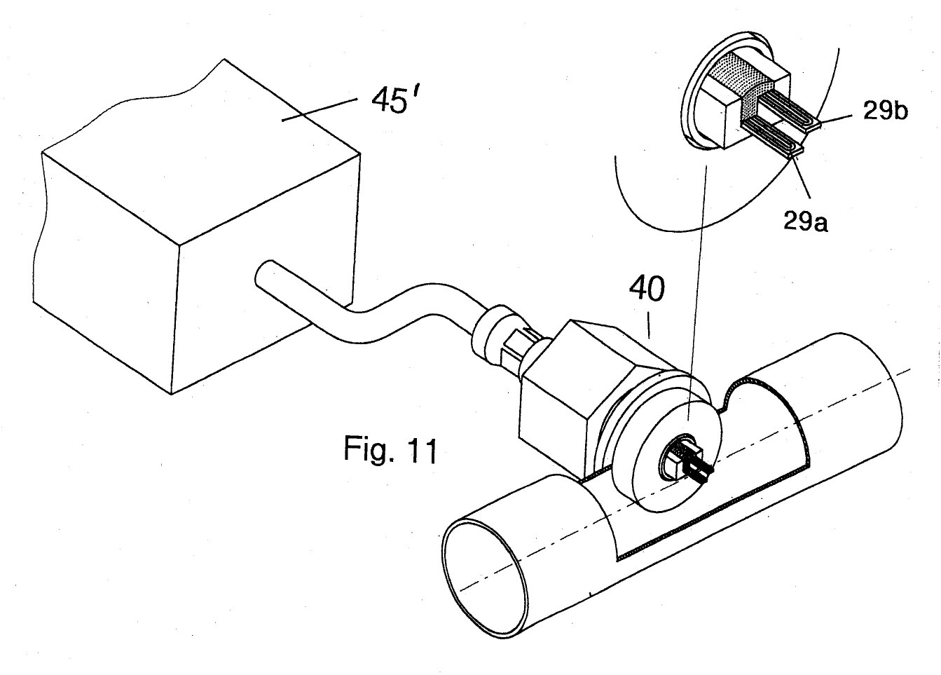

FIG. 10 represents the actual hot-film anemometer,

FIG. 11 shows, in a cut-out enlargement of the flow channel for the exhaust-gas recirculation with the actual measurement element,

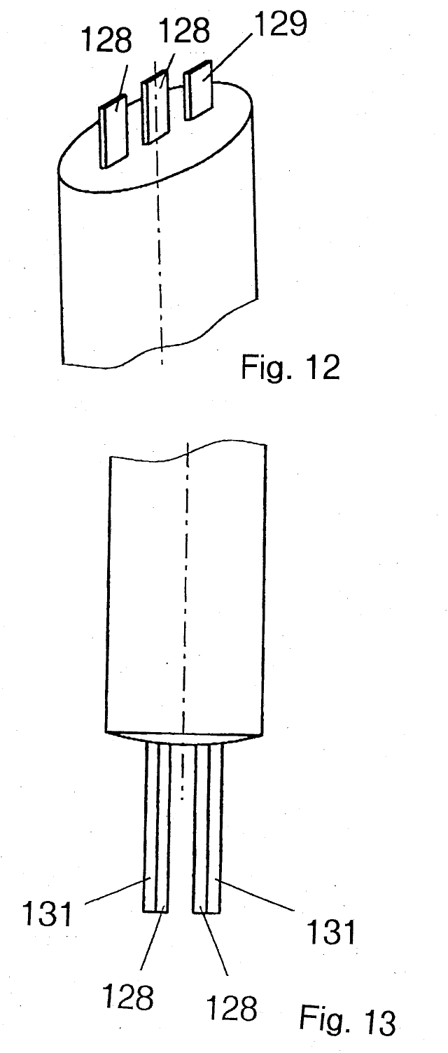

FIG. 12 shows a hot-film anemometer for identification of direction of flow,

FIG. 13 shows a hot-film anemometer with two thermally decoupled heaters,

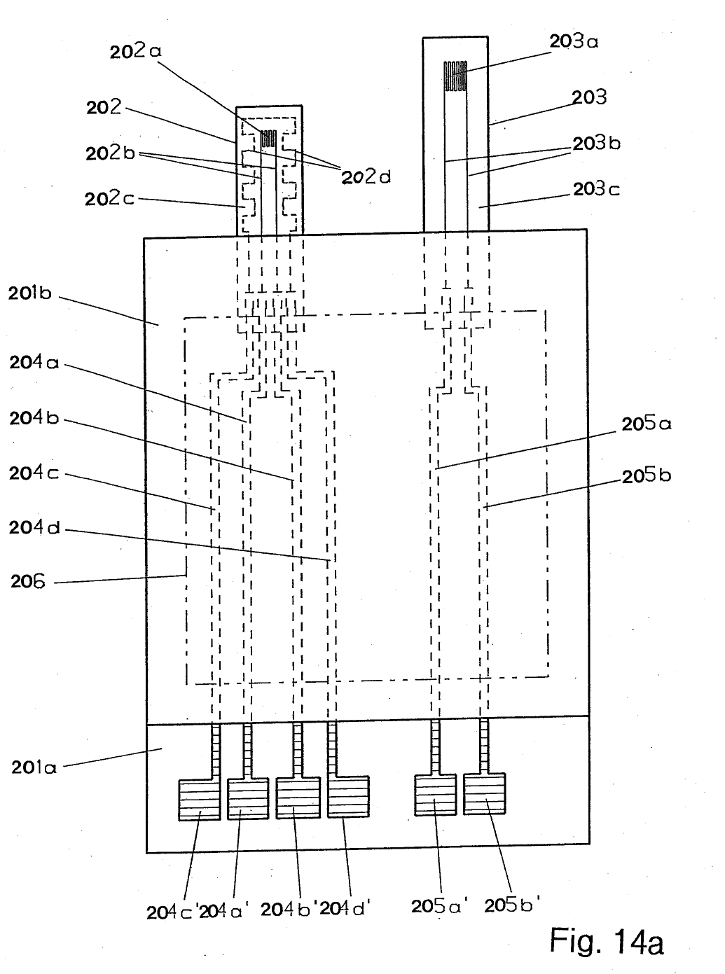

FIG. 14 shows a hot-film anemometer with two-layer ceramic-film laminate and a temperature measurement element and a heating element (top view of FIG. 14a),

FIG. 14a shows the hot-film anemometer from FIG. 14 in side view,

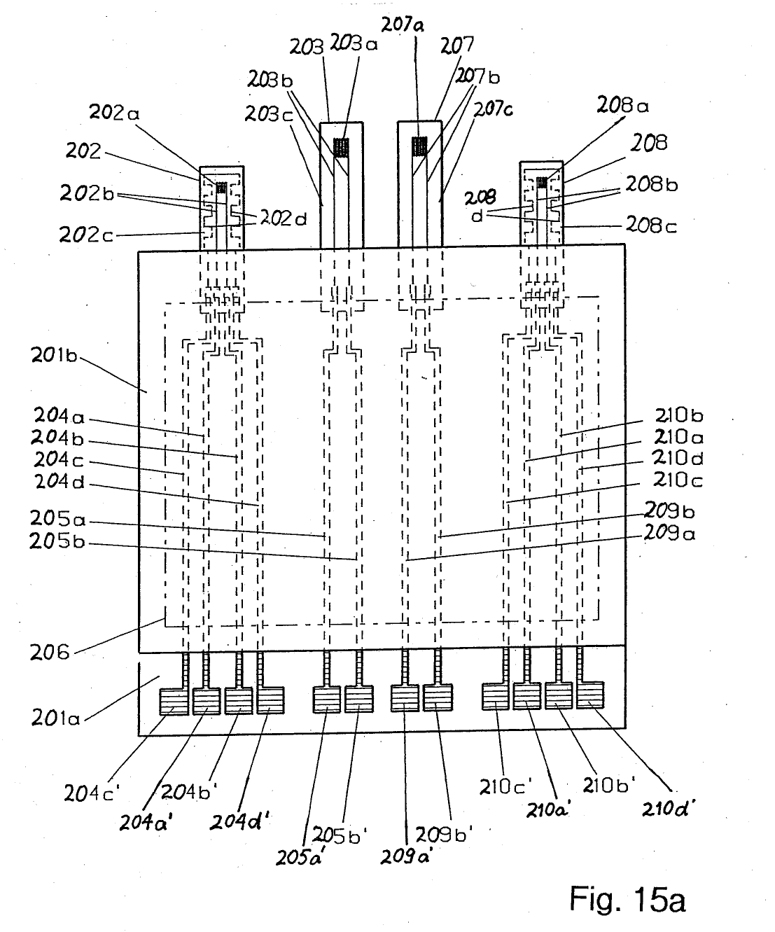

FIG. 15 shows a hot-film anemometer with two-layer ceramic-film laminate and two temperature measurement elements and two heating elements (top view of FIG. 5a-sic 15a),

FIG. 15a shows the hot-film anemometer from FIG. 15 in side view,

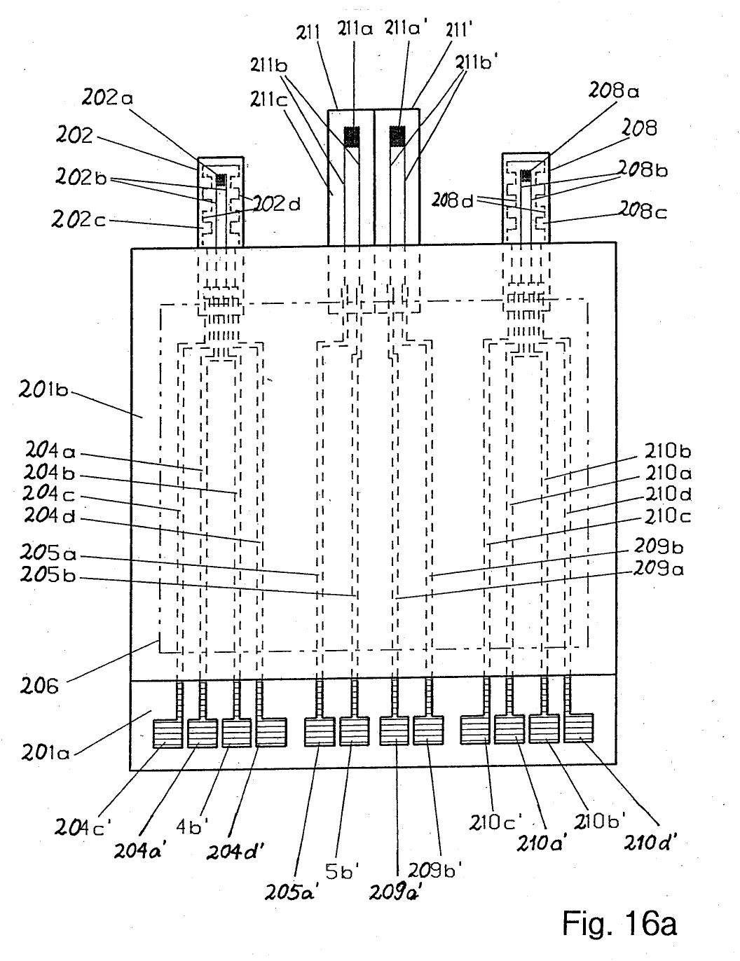

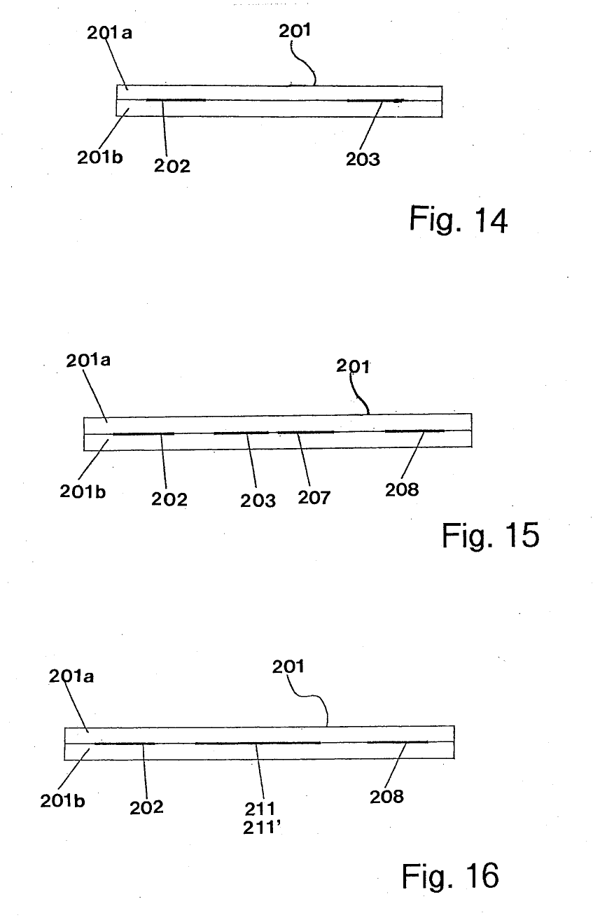

FIG. 16 shows a hot-film anemometer with two-layer ceramic film laminate and two temperature measurement elements and a double heating element (top view of FIG. 16a),

FIG. 16a shows the hot-film anemometer from FIG. 16 in side view,

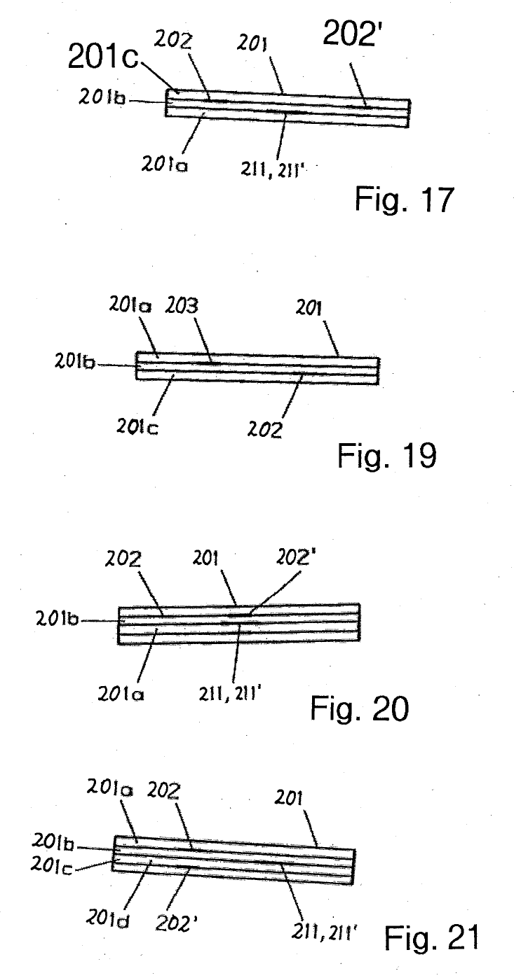

FIG. 17 shows a hot-film anemometer with three-layer ceramic film laminate, two temperature measurement elements, and a double heating element in top view,

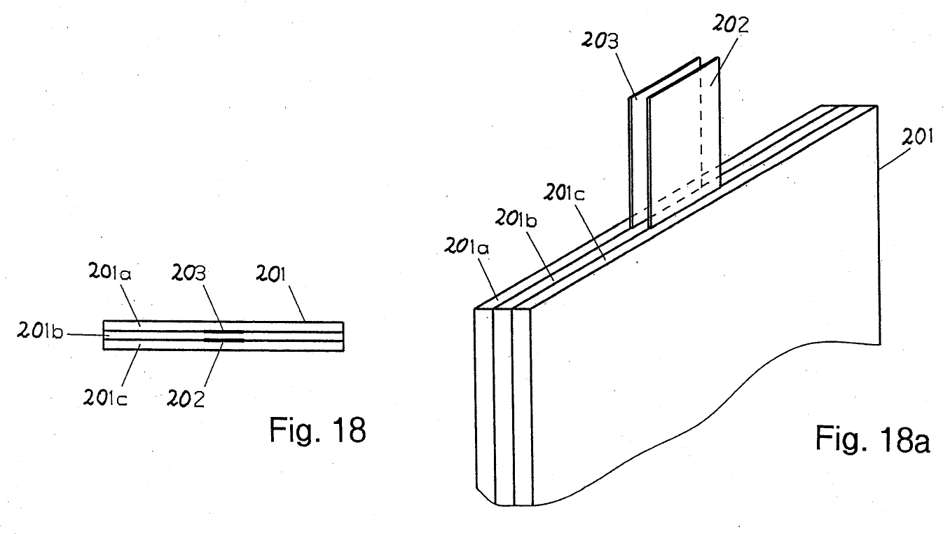

FIG. 18 shows a hot-film anemometer with three-layer ceramic film laminate, a temperature measurement element, and a heating element in top view,

FIG. 18a shows the hot-film anemometer from FIG. 18 in perspective view,

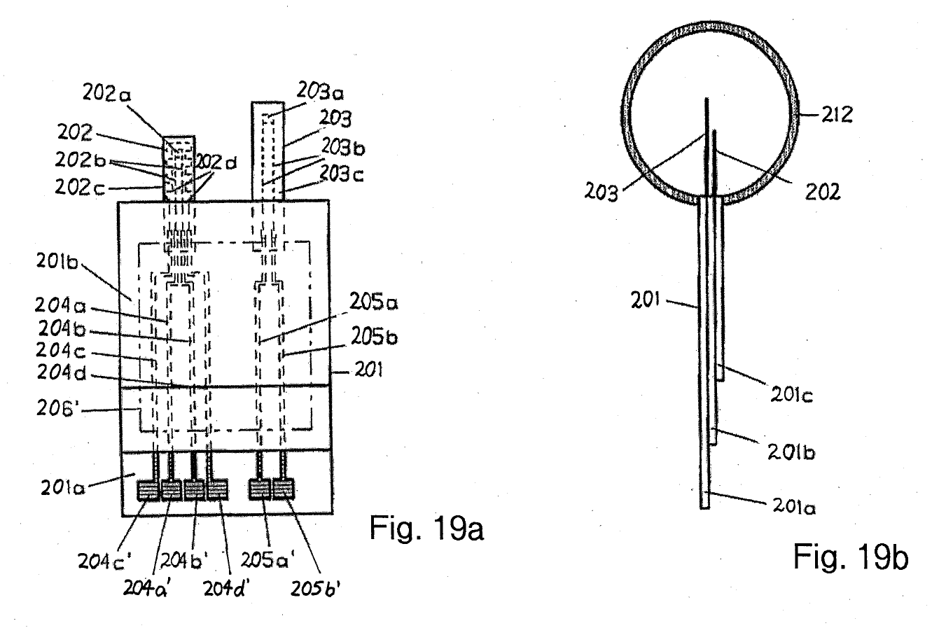

FIG. 19 shows a hot-film anemometer with three-layer ceramic film laminate, a temperature measurement element, and a heating element in top view,

FIG. 19a shows the hot-film anemometer from FIG. 19 in side view,

FIG. 19b shows the hot-film anemometer from FIG. 19a in side view,

FIG. 20 shows a hot-film anemometer with four-layer ceramic-film laminate, two temperature measurement elements, and a double heating element in top view,

FIG. 21 shows a hot-film anemometer with four-layer ceramic film laminate, two temperature measurement elements, and a double heating element in top view,

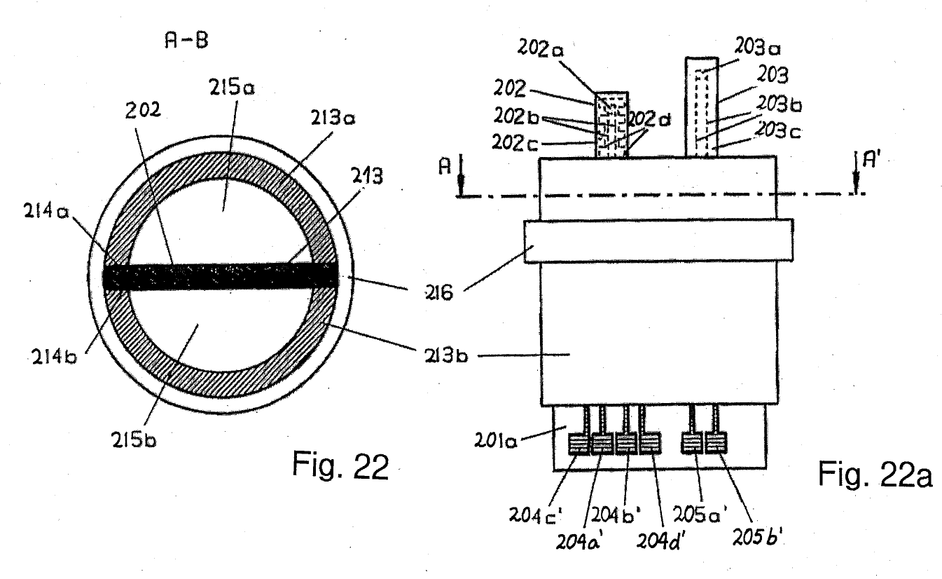

FIG. 22 shows a hot-film anemometer with multi-part ceramic component, a temperature measurement element, and a heating element in cross section A-A′ (see FIG. 12a [sic 22a]),

FIG. 22a shows the hot-film anemometer from FIG. 22 in side view,

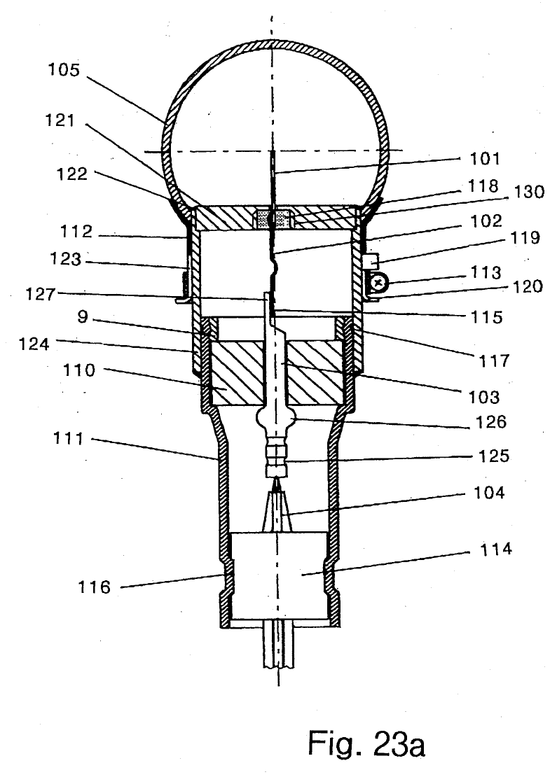

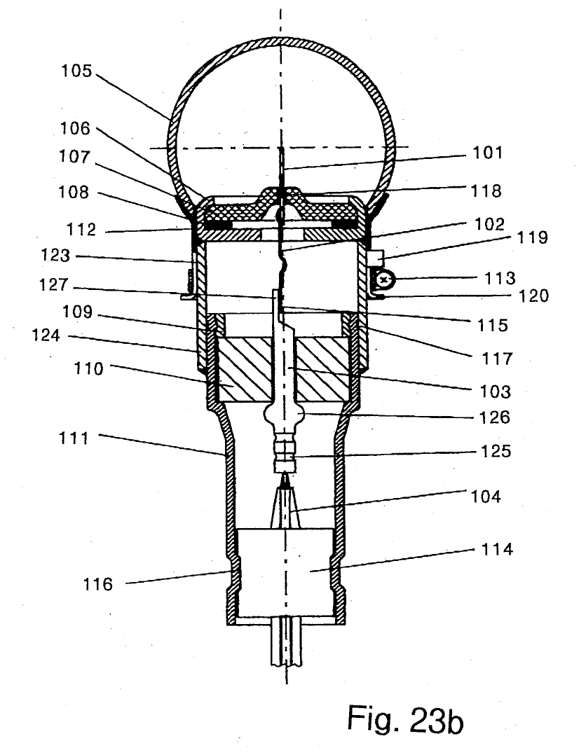

FIG. 23a shows a hot-film anemometer with heating and temperature measurement element arranged in a metal disk,

FIG. 23b shows a hot-film anemometer with heating and temperature measurement element arranged in a ceramic disk,

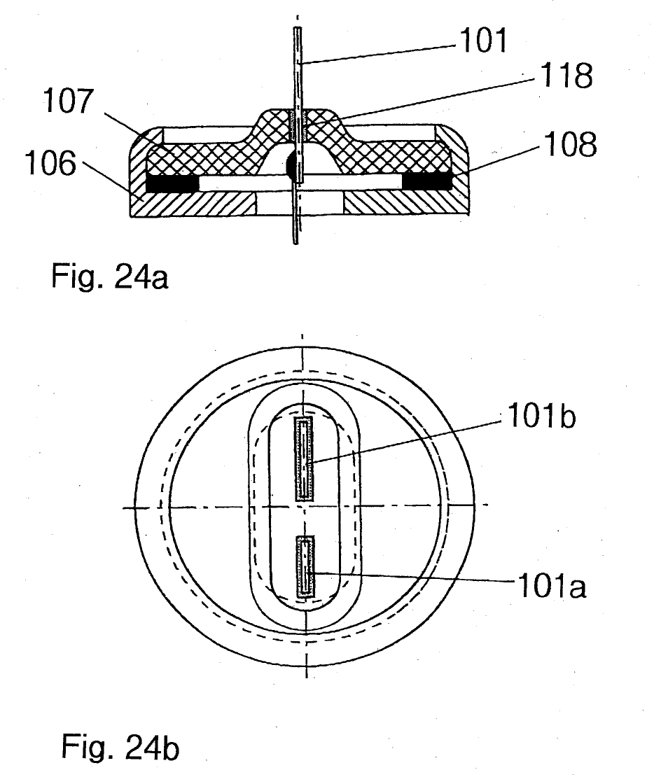

FIG. 24a shows a cutout according to FIG. 14 or 15 [sic-23b] relating to an arrangement of film resistors in a ceramic disk,

FIG. 24b shows the cutout according to FIG. 16a [sic-23b] in top view.

According to one simple anemometric measurement device according to FIG. 1 and FIG. 2, a film resistor 2 is fixed in a carrier 3 with glass solder or ceramic sealing compound. The sealing compound or glass solder holds the carrier air-tight to the substrate of the film resistor, in particular, in the region of the fixing drop [7]. The carrier 3 is sealed on the end opposite the measurement resistor against a metal housing 4a with a seal 5 made of an elastomer. The end of the carrier 3 sealed against the metal housing 4a is spaced apart during use at such a distance from the exhaust-gas pipe that this cool end of the anemometer is not heated so much during the operating condition that the elastomeric material used as a seal 5 loses its designed purpose. The metallic housing 4 is simultaneously a shield 4a against heat due to the air gap remaining between the metallic housing 4 and the carrier 3. An air-gap insulation between the metallic housing 4 and the carrier 3 is spaced with a spacer 6 that spaces, but does not seal, the housing 4 to the carrier 3 outside of the exhaust-gas pipe 8. This spacer 6 cools the carrier 3 heated in the exhaust-gas pipe interior by heat outflow outside of the exhaust-gas pipe 8. The film resistor 2 (chip) is partially covered in the region of the substrate with a cap 4c that has a slot, so that only the region of the measurement resistor of the film resistor projects significantly from the slot of the cap, in order to be arranged in the exhaust-gas flow. The fixing drop on the substrate and a part of the track conductors, which lead to the measurement resistor, are shielded thermally by the cap 4c.

The hot exhaust gas thus can diffuse, but not flow, through the slot of the cap into the gap between the carrier 3 and the shield 4 up to the seal 5. For this reason, the cold end of the anemometric measurement device spaced outside of the exhaust-gas pipe does not heat up so much that an elastomeric sealing material would be destroyed. The cold end is located between the housing parts 4b and 4c located outside of the exhaust-gas pipe. The housing part 4c forms the housing region 4c for the cable connection to the connection wires of the film resistor 2 that are guided through the carrier 3. The housing part 4b is required for sealing the carrier 3 against the housing 4.

FIG. 3 is a preferred embodiment of an anemometric measurement device with a film resistor 2 formed as a heater and a film resistor 2 formed as a temperature measurement element. Both film resistors 2 are respectively fixed with a sealing mass, for example, sealing compound, glass, or glass ceramic, in a carrier 3. The carrier 3 is sealed air-tight against the film resistors 2. Such a ceramic carrier 3 is shown in FIG. 5. The ceramic carriers 3 are formed as ceramic pipes, in particular ceramic multi-hole capillary pipes, and are each sealed on their end opposite the measurement resistor of the chip 2 with an elastomer mass 5 against a metallic housing 4. Between the carriers 3 and the metallic housing 4 there is an air gap and an air-permeable spacer 6. The spacer 6 is arranged so that it is spaced from the exhaust-gas pipe 8 outside of this pipe in the operating condition. The spacer 6 cools the ceramic carrier pipes 3 by heat dissipation to the housing 4.

The functional region of the film resistor 2 relevant for the measurement projects through slots of a cap 4a, which otherwise covers the film resistor.

Hot air outside of the film resistors can diffuse, but not flow, through slots of the cap 4c, which substantially covers the film resistors up to the seal of the ceramic carrier with the metal housing 4.

FIG. 4 shows the housing 4 sub-divided into the housing part 4a that projects into the exhaust-gas pipe and whose function is a covering cap 4a or thermal shield 4a. In this way, the carrier 3 is shielded from the heat of the medium. The part of the housing 4b arranged outside of the exhaust-gas pipe is spaced apart from the carrier 3 by spacers 6, wherein the spacers 6 according to FIG. 4 are formed as cooling ribs, in order to improve the cooling of the carrier 3 by heat flux via the spacers 6.

The housing part 4c surrounds the cable connection. Between the parts 4b and 4c of the housing, the seal 5 is arranged that holds the housing 4 sealed air-tight with an elastomer against the carrier 3. The elastomeric seal 5 is set in a holder 10.

FIG. 6 shows the cable output of the housing 4c in which the wires of a cable 11 are connected to the connection wires of the film resistors 2.

FIG. 7 shows a film resistor 2 on whose substrate 20, made of aluminum oxide ceramic, a track conductor 21 leads to a meander 22 that can be constructed as a measurement resistor or heating resistor. The connection wires 24 to the track conductors 21 are fixed with a fixing drop 23 made of glass or glass ceramic. The film resistor 2 is sealed and fixed with a glass solder 7 against the carrier 3. The cap 4a protects the seal and the carrier from thermal shocks and high thermal loading. The track conductor 21 electrically connects the meander 22, clearly located outside of the shield 4a due to the thermal decoupling, and the region of the film resistor 2 shielded by the shield 4.

According to FIG. 8, the internal combustion engine 31 has an inlet region 32 provided with charging for incoming air and recirculated exhaust gas; furthermore, on the exhaust-gas side of the engine there is an outlet region 34 from which a portion of the exhaust gas is led into a flow channel 36 for the exhaust-gas recirculation, wherein this channel is provided with a controllable valve 35, an exhaust-gas cooling device 38, as well as a hot-film anemometer 40. The hot-film anemometer 40 measures the quantity of the recirculated exhaust gas. The controllable valve 35 is used for the control of the exhaust gas partial pressure for the purpose of setting a specific quantity ratio between fresh, incoming air and the quantity of the partially recirculated exhaust gas; the controllable valve 35 is controlled with a regulator 45 by a control signal Y. The flow channel 36 for the exhaust-gas recirculation ends in an opening formed as a mixing chamber for introducing fresh air in the inlet region 32 of the engine 31, wherein, for measurement of incoming fresh air, an additional flow quantity sensor 44 is optional.

The portion of the exhaust gas branched in the recirculation to the flow channel 36 in the outlet region 34 of the engine 31 thus flows through the valve 35, cooling device 38, and hot-film anemometer 40 one after the other. In the opening, the recirculated exhaust gas meets the incoming air of the fresh air introduction after this has possibly passed through the optional mass flow sensor 44.

The mixture made of the incoming air and exhaust gas is fed to a charging device with a compressor that is advantageously formed as an exhaust-gas turbocharger, wherein the associated drive turbine is not shown in the outlet region 34 for the purpose of better clarity.

The portion of the exhaust gas not provided for recirculation is led via a line 50—and optionally a cleaning device—into open air, wherein the average temperature of the exhaust gas lies at ca. 400° C. to 700° C.

Furthermore, in the region of the exhaust-gas outlet region 34 there can be a temperature sensor for the measurement of the exhaust-gas temperature. The regulator 45 shown here symbolically receives via the line 41a signal X1 corresponding to the exhaust-gas temperature and via line 43 a signal Z corresponding to the quantity of incoming fresh air measured by the optional flow quantity sensor 44; because this quantity of incoming fresh air is normally not adjustable, this is represented by the symbol Z used for interference quantities in control technology.

FIG. 9 shows in longitudinal section the hot-film anemometer 40, wherein additional electrical connectors can be seen above the sensor housing shown in the longitudinal section. The inlet region is provided with the symbol 26; the outlet region is provided with the symbol 28.

In FIG. 10, the hot-film anemometer 40 is shown enlarged in longitudinal section, wherein, in the inlet region 26, a heatable measurement element 27, in particular a thin-film temperature sensor, can be seen with a heater, while in the outlet region 28 a heating element 29 can be seen.

In FIG. 11, the hot-film anemometer 40 is shown in a cutout of the flow channel 6 [delete—not correct] according to the two or multiple heater principle. In this figure, two heating elements 29a, 29b are to be seen as micro-heaters. The associated regulator is here provided with the symbol 15′ [sic 45′] and is formed as part of the engine control electronics, shown broken away.

In one embodiment of the hot-film anemometer, the heating element is constructed as a heat-output sensor and the temperature measurement element is constructed as a temperature sensor that additionally can carry a heat conductor for clean burning.

According to FIG. 12, for this purpose there are two heat output sensors 128 for detecting the direction of the media flow. The anemometric measurement principle basically functions so that the temperature measurement element detects the media temperature precisely. The heating element or the two heating elements of the heat output sensor or sensors 128 are then held at a constant excess temperature for the temperature sensor 129 by an electrical circuit. The gas or fluid flow to be measured cools the heating element or elements of the heat output sensor or sensors to a greater or lesser extent.

For maintaining the constant excess temperature, in the case of a mass flow, the electronics must deliver a corresponding current to the heating element or elements; on a precise measurement resistor, this generates a voltage that correlates with the mass flow and that can be evaluated. The double arrangement of the heat output sensor 128 or temperature sensor 129 here allows the detection of the direction of the mass flow.

In contrast, according to FIG. 13, in an embodiment as soot sensors, two heat output sensors are placed in a pipe housing lying parallel to each other.

The two heat output sensors 128 are here also each provided with a glazed on ceramic plate 131.

In the disclosed arrangement, a heat output sensor is operated above the pyrolytic incineration temperature; i.e., at ca. 500° C. The second heat output sensor is here operated in a lower temperature range of 200-450° C., preferably of 300-400° C. For soot deposits on this second heat output sensor, this deposited film acts as thermal insulation and change of the IR emission properties in the sense of an increasingly black body.

This can be evaluated electronically in a reference measurement for the first heat output sensor.

FIG. 14 shows a hot-film anemometer with a ceramic film laminate 201 formed from a first ceramic film 201a made of Al₂O₃ and a second ceramic film 201b made of Al₂O₃. A temperature measurement element 202 and a heating element 203 are partially embedded and electrically contacted in series between the first ceramic film 201a and the second ceramic film 201b. This allows a measurement of the mass flow rate according to the principle of the hot-film anemometer. The heating element 203 is here held either at a constant temperature (e.g. of 450° C.) or a constant temperature difference (e.g. of 100K) to the temperature measurement element 202 by an electrical control circuit (bridge circuit and amplifier in a control loop). A change in the mass flow of the medium now causes a change in the power consumption of the heating element 203, which can be evaluated electronically and stands in direct relation to the mass flow.

FIG. 14a shows the hot-film anemometer from FIG. 14 in side view. Here, it can be seen that the temperature measurement element 202 and the heating element 203 are electrically contacted by electrical track conductors 204a, 204b, 204c, 204d, 205a, 205b with connection surfaces 204a′, 204b′, 204c′, 204d′, 205a′, 205b′. The electrical track conductors 204a, 204b, 204c, 204d, 205a, 205b are arranged on the first ceramic film 201a and are partially covered by the second ceramic film 201b. Therefore, their position is shown partially with dashed lines. The temperature measurement element 202 has a carrier plate 202c comprising an individual film made of Al₂O₃. A platinum thin-film element 202a for the temperature measurement and 202d for heating and electrical connection lines 202b are arranged on the reverse side of the carrier plate 202c including an electrically insulating coating, and their positions are therefore shown with dashed lines. The heating element 203 has a carrier plate 203c comprising an individual film made of Al₂O₃. A platinum thin-film element 203a as a heater and its electrical connection lines 203b are arranged on the reverse side of the carrier film 203c and their positions are therefore shown with dashed lines.

The ceramic films 201a, 201b are connected in the region 206 either directly to each other through sintering or via a glass solder. The connection surfaces 204a′, 204b′, 204c′, 204d′, 205a′, 205b′ are uncovered by the second ceramic film 201b, so that a connection to electrical connection cables (not shown here) can be performed.

FIG. 15 shows a hot-film anemometer with a ceramic film laminate 201 formed from a first ceramic film 201a made of Al₂O₃ and a second ceramic film 201b made of Al₂O₃. Two temperature measurement elements 202, 208 and two heating elements 203, 207 are partially embedded and electrically contacted in series between the first ceramic film 201a and the second ceramic film 201b.

This allows, in turn, a measurement according to the principle of the hot-film anemometer, as already described for FIG. 14. The number of heating elements 203, 207 and temperature measurement elements 202, 208, however, now allows an electrical control loop to be formed and evaluated for each heating element and temperature measurement element (202 and 203 or 207 and 208). With this flow sensor element, it is now possible to detect the direction of flow of a medium, because thermal energy is transferred from the heating element arranged first in the direction of flow to the subsequent heating element. The change in temperature or heating of the subsequent heating element leads to a lower power consumption of this heating element, which can be evaluated as a signal for the direction of flow of the medium.

FIG. 15a shows the hot-film anemometer from FIG. 15 in side view. Here it can be seen that the temperature measurement elements 202, 208 and the heating elements 203, 207 are electrically contacted to connection surfaces 204a′, 204b′, 204c [c′], 204d [d′], 205a′, 205b′, 209a′, 209b′, 210a′, 210b′ [, 210c′, 210d′] via electrical track conductors 204a, 204b, 204c, 204d, 205a, 205b, 209a, 209b, 210a, 210b [, 210c, 210d]. The electrical track conductors 204a, 204b, 204c, 204d, 205a, 205b, 209a, 209b, 210a, 210b [, 210c, 210d] are arranged on the first ceramic film 201a and partially covered by the second ceramic film 201b. Therefore, their positions are shown partially with dashed lines. The temperature measurement element 202 has a carrier plate 202c comprising two individual layers made of Al₂O₃ and SiO₂.

A platinum thin-film element 202a for temperature measurement and 202d for annealing and its electrical connection lines 202b are arranged on the reverse side of the carrier plate 202c and therefore their positions are shown with dashed lines. The heating element 203 has a carrier plate 203c comprising two individual layers made of Al₂O₃ and SiO₂. A platinum thin-film element 203a as a heater and its electrical connection lines 203b are arranged on the reverse side of the carrier plate 203c and therefore their positions are shown with dashed lines. The heating element 207 has a carrier plate 207c comprising two individual layers made of Al₂O₃ and SiO₂. A platinum thin-film element 207a as a heater and its electrical connection lines 207b are arranged on the reverse side of the carrier plate 207c and therefore their positions are shown with dashed lines. The temperature measurement element 208 has a carrier plate 208c comprising two individual layers made of Al₂O₃ and SiO₂. A platinum thin-film element 208d for temperature measurement and 202a for heating and its electrical connection lines 208b are arranged on the reverse side of the carrier plate 208c and therefore their positions are shown with dashed lines.

The ceramic films 201a, 201b are connected in the region 206 either directly to each other through sintering or via a glass solder. The connection surfaces 204a′, 204b′, 204c′, 204d′, 205a′, 205b′, 209a′, 209b′, 210a′, 210b′, 210c′, 210d′ are uncovered by the second ceramic film 201b, so that a connection to the electrical connection cables (not shown here) can be performed.

FIG. 16a shows the hot-film anemometer from FIG. 16 in side view. Here, it can be seen that the temperature measurement elements 202, 208 and the double heating element 211, 211′ are electrically contacted to connection surfaces 204a′, 204b′, 204c′, 204d′, 205a′, 205b′, 209a′, 209b′, 210a′, 210b′, 210c′, 210d′ via electrical track conductors 204a, 204b, 204c, 204d, 205a, 205b, 209a, 209b, 210a, 210b, 210c, 210d. The electrical track conductors 204a, 204b, [204c, 204d,] 205a, 205b, 209a, 209b, 210a, 210b, 210c, 210d are arranged on the first ceramic film 201a and are partially covered by the second ceramic film 201b. Therefore, their positions are shown partially with dashed lines. The temperature measurement element 202 has a carrier plate 202c comprising an individual layer made of Al₂O₃. A platinum thin-film element 202a for temperature measurement and 202d for heating and its electrical connection lines 202b are arranged on the reverse side of the carrier plate 202c including an electrically insulating coating and therefore their positions are shown with dashed lines. The double heating element 211, 211′ has a carrier plate 211c comprising two individual layers made of Al₂O₃ and SiO₂. Platinum thin-film elements 211a, 211a′ as heaters and their electrical connection lines 211b, 211b′ are arranged on the reverse side of the carrier plate 211c including an electrically insulating coating and therefore their positions are shown with dashed lines. The temperature measurement element 208 has a carrier plate 208c comprising two individual layers made of Al₂O₃ and SiO₂. A platinum thin-film element 208d for temperature measurement and 208a for heating and its electrical connection lines 208b are arranged on the reverse side of the carrier plate 208c and therefore their positions are shown with dashed lines.

In the region 206, the ceramic films 201a, 201b are sintered directly to each other or connected by glass solder. The connection surfaces 204a′, 204b′, 204c′, 204d′, 205a′, 205b′, 209a′, 209b′, 210a′, 210b′, 210c′, 210d′ are uncovered by the second ceramic film 201b, so that a connection to electrical connection cables (not shown here) can be performed.

FIG. 17 shows a hot-film anemometer with a ceramic film laminate 201 formed from a first ceramic film 201a made of Al₂O₃, a second ceramic film 201b made of Al₂O₃, and a third ceramic film 201c made of Al₂O₃. Between the second ceramic film 201b and the third ceramic film 201b [sic 201c], two temperature measurement elements 202, 202′ are partially embedded and electrically contacted. Between the first ceramic film 201a and the second ceramic film 201b, a double heating element 211, 211′ is partially embedded and electrically contacted. Here, a double heating element is understood to be two heating elements that can be controlled separately electrically and that are constructed on a common carrier plate. With this flow sensor element, it is also possible to detect the direction of flow of a medium.

FIGS. 18 and 18a and FIG. 19 each show a hot-film anemometer with a ceramic film laminate 201 formed from a first ceramic film 201a, a second ceramic film 201b, and a third ceramic film 201c made of Al₂O₃. Between the first ceramic film 201a and the second ceramic film 201b, a temperature measurement element 202 [sic—heating element 203] is partially embedded and electrically contacted. Between the second ceramic film 201b and the third ceramic film 201c, a heating element 203 [sic—temperature measurement element 202] is partially embedded and electrically contacted. With these flow sensor elements it is not possible to detect the direction of flow of a medium.

FIG. 19a shows the flow sensor element from FIG. 19 in side view. Here it can be seen that the temperature measurement element 202 and the heating element 203 are electrically contacted to connection surfaces 204a′, 204b′, [204c′, 204d′,] 205a′, 205b′ via electrical track conductors 204a, 204b, [204c, 205d,] 205a, 205b. The electrical track conductors 205a, 205b are arranged on the first ceramic film 201a and partially covered by the second ceramic film 201b. Therefore, their positions are shown partially with dashed lines. The electrical track conductors 204a, 204b [204c, 204d,] are arranged on the second ceramic film 201b and partially covered by the third ceramic film 201c [not labeled]. Therefore, their positions are shown partially with dashed lines. The temperature measurement element 202 has a carrier film 202c comprising an individual layer made of Al₂O₃. A platinum thin-film element 202a for temperature measurement and its electrical connection lines 202b are arranged on the reverse side of the carrier plate 202c including an electrically insulating coating and their positions are shown with dashed lines. In a preferred embodiment, the carrier plate is equipped with an additional thin-film element 202d for heating the temperature element that is contacted electrically analogously. The heating element 20 [sic—203] has a carrier plate 203c comprising an individual layer made of Al₂O₃. A platinum thin-film element 203a as a heater and its electrical connection lines 203b are arranged on the reverse side of the carrier plate 203c and therefore their positions are shown with dashed lines. The ceramic films 201a, 201b [201b in FIG. 19a should be 201c, and the intermediate length rectangle ending below 206′ should be 201b, as shown in FIG. 19b] are connected in the region 206′ either directly to each other through sintering or via a glass solder. The connection surfaces 5a′, 5b′ [sic—205a′, 205b′] are uncovered by the second ceramic film 1b [sic—201b], so that a connection to electrical connection cables not shown here can be performed. The ceramic films 1b, 1c [sic—201b, 201c] are connected in the region 206 [not labeled] either directly to each other through sintering or via a glass solder. The connection surfaces 204a′, 204b′ [, 204c′, 204d′] are uncovered by the third ceramic film 201c [mislabeled 201b], so that a connection to electrical connection cables (not shown here) can be performed.

FIG. 19b shows the hot-film anemometer from FIG. 19a in side view, wherein this is installed in the cross section of a pipe line 212. The carrier films 202c, 203c of the temperature measurement element 202 and the heating element 203 are here inserted into the pipe line parallel to the direction of flow.

FIG. 20 [FIG. 20 doesn't show this and doesn't belong here] and FIG. 21 each show a hot-film anemometer with a ceramic film laminate 201 formed from a first ceramic film 201a, a second ceramic film 201b, a third ceramic film 201c, and a fourth ceramic film 201d [lead line misplaced in FIG. 21] made of Al₂O₃. Between the first ceramic film 201a and the second ceramic film 201b, a temperature measurement element 202 is partially embedded and electrically contacted. Between the second ceramic film 201b and the third ceramic film 201c, a double heating element 211, 211′ is partially embedded and electrically contacted. Between the third ceramic film 201c and the fourth ceramic film 201d, another temperature measurement element 202′ is partially embedded and electrically contacted.

FIG. 22 [mislabeled A-B in FIG. 22] shows a hot-film anemometer in cross section A-A′ (see FIG. 22a) with a multiple-part ceramic component 213a, 213b, 214a, 214b made of Al₂O₃, which has a temperature measurement element 202 and a heating element 203 [mislabeled 213 in FIG. 22]. The ceramic component 213a, 213b, 214a, 214b has two hollow spaces 215a, 215b that are closed gas-tight in the region of the temperature measurement element 202 and the heating element 203, respectively. For installation, a connection flange 216 is present in a pipe line.

FIG. 22a shows the hot-film anemometer from FIG. 22 in side view. Here, the temperature measurement element 202 and the heating element 203 are electrically contacted to connection surfaces 204a′, 204b′, [204c′, 204d′,] 205a′, 205b′ via electrical track conductors 204a, 204b, [204c, 204d,] 205a, 205b that can be seen here only partially. The electrical track conductors 204a, 204b, [204c, 204d,] 205a, 205b are arranged on a ceramic plate 214a and—not visible in this view—partially covered by a second ceramic plate 214b. The temperature measurement element 202 has a carrier plate 202c comprising an individual layer made of Al₂O₃. A platinum thin-film element 202a for temperature measurement and its electrical connection lines 202b are arranged on the reverse side of the carrier film 202c and therefore their positions are shown with dashed lines. In a preferred embodiment, the carrier plate 202c has an additional platinum thin-film element 202d with a resistance that is smaller by one order of magnitude. This resistor designed for heating or annealing is electrically contacted to an additional contact analogously to the thin-film element 202a. The heating element 203 has a carrier plate 203c comprising an individual layer made of Al₂O₃. A platinum thin-film element 203a as a heater and its electrical connection lines 203b are arranged on the reverse side of the carrier plate 203c and therefore their positions are shown with dashed lines.

The ceramic plates 214a, 214b are connected either directly to each other through sintering or to each other via a glass solder and to pipe shells 213a, 213b to form the ceramic component. However, it is also possible to use two half pipes (213a plus 214a; 213b plus 214b) in which the ceramic plate 214a and the pipe shell 213a or the ceramic plate 214b and the pipe shell 214b are each combined into a one-piece component. The connection surfaces 204a′, 204b′, [204c′, 204d′,] 205a′, 205b′ are uncovered by the second ceramic plate 214b, so that a connection to electrical connection cables (not shown here) can be performed.

According to FIG. 23a, a hot-film anemometer is made with sealing compound or glass 118 in a carrier disk 121 made of heat-resistant and exhaust-gas-resistant stainless steel. By a structured inner wall of the sealing compound space, e.g., by a thread 130, a good grip of the sealing compound is achieved. The region of the carrier disk 121 through which the sensor element projects toward the medium has rectangular contours that are only slightly greater than the sensor element cross section.

Therefore, the hot-film anemometer is held directed into the media-guiding pipe 105, and the inner space of the complete sensor is sealed against the medium.

The carrier disk 121 is inserted into a housing [pipe] 124 and fused tightly with a round seam 122. In the housing pipe 124, the housing 111 is fused. In the housing 111 the insulating body 110, made of temperature-resistant plastic or ceramic, is held with a ring 109 [sic—9 in FIG. 23a ] that is fixed by a bead 117. On the cable outlet, with the bead 116, a cable grommet made of an elastomer is fixed tightly. Feed lines 104 are guided through the through holes of a grommet 114. Each feed line is electrically connected to a contact sleeve 103 by a crimp 125. The contact sleeve 103 has, under an insulating part 110, a wide section 126 and, above the insulating part 110, a surface 127 that is wider than the contact-sleeve diameter, so that the contact sleeve is fixed in the axial direction in the insulating part 110. On the surface 127, the connection wires 102 are electrically contacted with the weld 115.

The attachment of the complete sensor to the media-guiding pipe 105 is realized by a typical screw thread pipe shell 113 and a slotted plate flange part 112 welded on the media-guiding pipe 105.

The orientation of the hot-film anemometer 101 in the pipe 105 is realized by a centering pin 119 fixed on the housing pipe 124 and by the wide slot 120 in the plate flange part 112. Opposite a wide slot 120, there is a narrow slot 123 that only serves to be able to press the plate flange part 112 more easily onto the housing pipe 124. Thus, assembly is permitted only at the correct angle position.

FIG. 23b [along with FIGS. 24a and 24b ] shows another embodiment with a ceramic carrier disk 107, in which the flow element 101 is fixed with glass solder 118 in the carrier disk 107. The carrier disk 107 is flanged together with a high temperature-resistant seal 108 made of mica or graphite in the metallic holder 106. The holder 106 is also fused tightly to the housing pipe 124.

1. Arrangement of a film resistor (chip)(2) in an exhaust-gas pipe or exhaust-gas recirculation pipe, wherein the film resistor (2) is fixed in a carrier (3), which is sealed against a shield (4a) or a housing (4b), wherein the shield or housing is connected tightly to the exhaust-gas pipe or exhaust-gas recirculation pipe, characterized in that the carrier (3) and the shield or the housing are sealed against each other spaced apart from the exhaust-gas pipe or exhaust-gas recirculation pipe radially outside of this pipe.

2. Arrangement according to claim 1, characterized in that the film resistor (2) of a measurement device (1) projects into the exhaust-gas pipe or exhaust-gas recirculation pipe, wherein the measurement device comprises the carrier and the shield or the housing, as well as the seal (5) between the carrier (3) and the shield (4a) or the housing (4b).

3. Arrangement of a film resistor (2) in an exhaust-gas pipe or exhaust-gas recirculation pipe, wherein the film resistor (2) is fixed in a carrier (3), in particular according to claim 1, characterized in that the carrier (3) is sealed against a shield (4a) or a housing (4b) with an elastomeric seal (5).

4. Arrangement according to claim 1, characterized in that the carrier (3) is fixed in the metallic housing (4b) outside of the exhaust-gas or exhaust-gas recirculation pipe.

5. Arrangement according to claim 1, characterized in that the film resistor (2) is sealed gas-tight relative to the carrier (3) with an inorganic material.

6. Measurement device (1), in particular for an arrangement according to claim 1, containing a film resistor (2), a carrier (3), in particular a hollow body, in which the film resistor is fixed mechanically, and a shield (4a) or a metallic housing (4b), wherein this shield or housing is sealed against the carrier, characterized in that the electrical feed lines for the film resistor are arranged in the longitudinal direction of the carrier and the film resistor is spaced apart from the seal (5) along the carrier length, wherein this seal seals the carrier tightly against the shield (4a) or the housing (4b).

7. Measurement device according to claim 6, characterized in that the shield (4a) has at least one feedthrough around at least one film resistor and, moreover, encloses the carrier (3) in the region of the film resistor (2).

8. Arrangement of a measurement device, in particular according to claim 6, in an exhaust-gas pipe or exhaust-gas recirculation pipe, characterized in that the anemometric measurement device projects with a film resistor (2) into the exhaust-gas pipe or exhaust-gas recirculation pipe and the carrier (3) of the film resistor is led through the exhaust-gas pipe or exhaust-gas recirculation pipe and is sealed on its side located outside of the exhaust-gas pipe or exhaust-gas recirculation pipe against a metallic housing (4b).

9. Method for the production of an anemometric measurement device, comprising a heating element for the anemometric measurement that optionally also has one or two measurement resistors and optionally an additional measurement resistor that optionally also has a heating resistor for self cleaning, wherein the film resistor constructed as a heating element is fixed in a carrier, in particular hollow body, whose coefficient of expansion differs from that of the film resistor by no more than 2.5×10⁻⁶/K, in particular by a maximum of 1×10⁻⁶/K, characterized in that the carrier is fixed in a metallic housing, so that it remains spaced apart from the metallic housing outside of the attachment.

10. Use of a metallic housing (4) for thermal insulation of a measurement device (1) against hot exhaust gases, characterized in that the metallic housing is spaced apart by a carrier (3), in particular a hollow body, in its region projecting into the exhaust-gas pipe by a film resistor.

11. Use of a measurement device according to claim 1, as an anemometric measurement device or as a soot sensor or as a temperature sensor.

12. Measurement method with a film resistor (2) within a hot fluid greater than 500° C., wherein the film resistor (chip) is fixed in a carrier (3), which is sealed against a metal housing (4b) outside of the metal pipe, characterized in that the metal housing is connected tightly to the metal pipe.

13. Device, in particular for an arrangement according to claim 1, for the exhaust-gas recirculation from an outlet region (34) of a vehicle internal combustion engine (31) into an air inlet region (32), to which can be fed an adjustable mixture made of exhaust gas and incoming air of the engine (31), and a fuel quantity is adjustable, wherein the outlet region (34) is connected to an inlet region (32) of the internal combustion engine (1) via an exhaust-gas recirculation line having a controllable valve (35), an exhaust-gas cooling device (38), and a hot-film anemometer (40), wherein the hot-film anemometer (10—sic 40) has a heating resistor (29, 128, 203, 207) and a temperature measurement resistor (27, 129, 202, 208), the heating resistor (29, 129, 203, 207) and the temperature measurement resistor (27, 202, 129, 208) are separate film resistors (128, 129, 202, 203, 207, 208, 211), characterized in that the temperature measurement resistor (27, 129, 202, 208) and the heating resistor (29, 128, 203, 207) are each fixed to a ceramic carrier (7, 201).

14. Device, in particular according to claim 13, for the exhaust-gas recirculation from an outlet region (34) of a vehicle internal combustion engine (31) into an air inlet region (32), to which can be fed an adjustable mixture made of exhaust gas and incoming air of the engine (31), and a fuel quantity is adjustable, wherein the outlet region (34) is connected to an inlet region (32) of the internal combustion engine (31) by an exhaust-gas recirculation line having a controllable valve (35), an exhaust-gas cooling device (8 [sic 38]), and a hot-film anemometer (40), wherein the hot-film anemometer (40) has a heating resistor (29) and a temperature measurement resistor (27, 202, 129, 208), the heating resistor (29, 128, 203, 207), and the temperature measurement resistor (27, 202, 129, 208) are separate film resistors, characterized in that the hot-film anemometer (40) is arranged in an exhaust-gas recirculation channel in front of the cooling system (8) or in an air-cooled cooler or that a hot-film anemometer (40) is not arranged for either the fresh air or for the cooled exhaust gas.

15. Device according to claim 13, characterized in that its hot-film anemometer (40) has an opening of a cover (107) or a hollow body surface, in which are fixed a temperature measurement element (27, 129) and a heating element (29, 128).

16. Method for the self cleaning of a hot-film anemometer (40) of a device according to claim 13, in which a temperature measurement element (27, 129, 202, 208) and a heating element (29, 128, 203, 207) are arranged on a common carrier element (107, 201) or are fixed in an opening of a cover (107) or a hollow body surface, characterized in that the temperature measurement element (27, 202, 129, 208) has a platinum-thin-film resistor (202a) on a ceramic substrate (202c) for temperature measurement and is heated with an additional platinum-thin-film resistor (202d).

17. Method for the production of a hot-film anemometer (40) of a device according to claim 13, made of film resistors and a cover (107) or a hollow body, wherein at least two film resistors, whose resistances differ by one to two orders of magnitude, are placed in openings of the cover (107) or hollow body and are fixed in the openings or in which a temperature measurement element (27, 129, 202, 208) and a heating element (29 128, 203, 207) are arranged on a carrier element (107, 201) and the carrier element (201) is laminated from ceramic films (201a, 201b, 201c).
